(19) United States
(12) Patent Application Publication
(10) Pub. No.: US 2022/0268782 A1
(43) Pub. Date: Aug. 25, 2022

(54) METHODS FOR PREDICTING AND TREATING CARDIAC DYSFUNCTION

(71) Applicants: INSERM (INSTITUT NATIONAL DE LA SANTÉ EAT DE LA RECHERCHE MÉDICALE), Paris (FR); ASSISTANCE PUBLIQUE-HÔPITAUX DE PARIS (APHP), Paris (FR); UNIVERSITÉ PARIS-EST CRÉTEIL VAL DE MARNE, Cret (FR)

(72) Inventors: Daigo Sawaki, Creteil (FR); Gabor Czibik, Creteil (FR); Geneviève Derumeaux, Creteil (FR)

(21) Appl. No.: 17/627,351

(22) PCT Filed: Jul. 13, 2020
(86) PCT No.: PCT/EP2020/069693

§ 371 Date: Jan. 14, 2022

(30) Foreign Application Priority Data
Jul. 15, 2019 (EP) 19186370.3

(51) Int. Cl. G01N 33/68

(52) U.S. Cl. CPC G01N 33/6812; G01N 2800/32

(57) ABSTRACT

Ageing myocardium undergoes structural and functional changes characterized by progressive cardiomyocyte hypertrophy, interstitial fibrosis and inflammation ultimately leading to diastolic and systolic dysfunction. Whilst most focus has been placed on established risk factors such as dyslipidaemia, hypertension and obesity in accelerating cardiac ageing, a potential role for amino acids has received little attention. Here the inventors show that increased phenylalanine (PA) levels induced in vitro cytosolic oxidative stress and senescence whilst in vivo led to senile-like cardiac deterioration in young mice. Moreover, they demonstrated that hepatic PA catabolism declined with age in a p21-dependent manner, whilst p21 deficiency prevented age-related cardiac dysfunction. Finally, the inventors found that Pah cofactor BH4 reversed the age-related rise in plasma PA levels and senile cardiac alterations. These observations have immediate implications for promoting cardiac health and healthspan and suggest that phenylalanine can be used as a biomarker and biotarget of cardiac dysfunction.

### FIELD OF THE INVENTION

The present invention is in the field of cardiology.

### BACKGROUND OF THE INVENTION

Ageing myocardium undergoes structural and functional changes characterized by progressive cardiomyocyte hypertrophy, interstitial fibrosis and inflammation ultimately leading to diastolic and systolic dysfunction.¹⁻³ Whilst most focus has been placed on established risk factors such as dyslipidaemia, hypertension and obesity in accelerating cardiac ageing,⁴ a potential role for amino acids has received little attention. Several lines of evidence drew our interest to the impact of high plasma phenylalanine (PA) levels on cardiac ageing. Firstly, a recent metabolomic study found negative correlation of PA levels with leukocyte telomere length in the elderly⁵ suggesting that increased levels of this essential amino acid may promote bodywide senescence. Secondly, elevated plasma PA levels predict heart failure, raising the hypothesis that PA is cardiotoxic.⁶ Moreover, transcript levels of the BH4-dependent rate-limiting enzyme in PA catabolism phenylalanine hydroxylase (Pah), whose expression is physiologically confined to liver and kidney,⁷ is progressively induced in ageing murine hearts.⁸ However, the role of increased PA levels in cardiac senescence and dysfunction is unknown.

### SUMMARY OF THE INVENTION

As defined by the claims, the present invention relates to methods for predicting and treating cardiac dysfunction.

### DETAILED DESCRIPTION OF THE INVENTION

Here the inventors show that increased PA levels induced in vitro cytosolic oxidative stress and senescence whilst in vivo led to senile-like cardiac deterioration in young mice. Moreover, they demonstrated that hepatic PA catabolism declined with age in a p21-dependent manner, whilst p21 deficiency prevented age-related cardiac dysfunction. Finally, the inventors found that Pah cofactor BH4 reversed the age-related rise in plasma PA levels and senile cardiac alterations. These observations have immediate implications for promoting cardiac health and healthspan.

The present invention relates to a method of predicting whether a subject has or is at risk of having a cardiac dysfunction comprising determining the level of phenylalanine in sample obtained from the subject wherein said level indicates whether the subject has or is at risk of having a cardiac dysfunction.

As used herein, the term “cardiac dysfunction” also referred to as “myocardial dysfunction” is known by the person skilled in the art. The term relates to any kind of heart dysfunction, more particularly, the term relates to heart dysfunction affecting the pumping capability of the heart. In particular, the term “cardiac dysfunction” relates to a condition in which myocardial contractility, metabolism and ventricular function are reduced in order to cope with a reduced oxygen supply. Typically, cardiac dysfunction involves cardiac remodeling and/or cardiac fibrosis and/or impairment of systolic function (left ventricular ejection fraction (LVEF) function or strain) and/or impairment of diastolic dysfunction. Cardiac dysfunction may be asymptomatic and can occur out of any heart failure symptoms. According to the invention, cardiac dysfunction is related to cardiac senescence.

As used herein, the term “cardiac senescence” has its general meaning in the art and refers to cardiac ageing manifesting as a decline in function ultimately leading to heart failure. Cardiac senescence can be characterized by both quantitative alterations, e.g., a decrease in the number of cardiomyocytes with age, and qualitative alterations, e.g., changes in cardiomyocyte properties and extracellular matrix remodeling with age. (31) At the cellular level, ageing entails dysregulation of cellular processes in myocytes and nonmyocytes. Senescent hearts are characterized by prolonged relaxation, diminished contraction velocity, decreased β-adrenergic response, and increased myocardial stiffness. This impairment in diastolic function contributes to the increased incidence of heart failure and atrial fibrillation in the elderly patients (31). In addition to endogenous dysfunction, senescent cells become pathogenic in most cases by mediating chronic sterile inflammation and tissue remodeling. In addition, the accumulation of myocardial collagen and extracellular matrix increases with age, contributing to myocardial stiffness and cardiac diastolic dysfunction.

The phenomena of cellular senescence can be accelerated by various factors (such as oxidative stress, metabolic factors and genotoxic agents). In this case, we speak of “premature senescence”.

In some embodiment, the cardiac dysfunction is cardiac senescence.

Thus, in some embodiment, the invention refers to a method of predicting whether a subject has or is at risk of having cardiac senescence comprising determining the level of phenylalanine in sample obtained from the subject wherein said level indicates whether the subject has or is at risk of having an early cardiac senescence.

In some embodiment, the cardiac senescence is premature cardiac senescence.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no- conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

In some embodiments, the subject can be male or female.

In some embodiments, the subject can be one who exhibits one or more risk factors for cardiac dysfunction (e.g. age, alcohol consumption, cigarette smoking, metabolic syndrome, obesity, diabetes/insulin resistance, hypertension, dyslipidaemia, liver disease, chronic kidney disease) or a subject who does not exhibit risk factors, or a subject who is asymptomatic for cardiac dysfunction (e.g. in case of a screening test).

In some embodiments, the subject is an elderly subject. As used herein, the term “elderly subject” refers to an adult patient sixty-five years of age or older.

In some embodiments, the subject is obese. The term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m2 to less than 25 kg/m².

In some embodiments, the subject suffers from a form of acquired and hereditary hyperphenylalaninemia, including phenylketonuria (PKU).

As used herein the term “sample” refers to any biological sample obtained from the subject that is liable to contain phenylalanine. Typically, samples include but are not limited to body fluid samples, such as blood, ascites, urine, amniotic fluid, feces, saliva or cerebrospinal fluids. In some embodiments, the sample is a blood sample. By “blood sample” it is meant a volume of whole blood or fraction thereof, e.g., serum, plasma, etc.

As used herein, the term “phenylalanine” or “PA” has its general meaning in the art and refers to the compound having the IUPAC name of (S)-2-Amino-3-phenylpropanoic acid.

According to the present invention, the level of phenylalanine may be determined by any routine method well known in the art. Typically, the level is determined as described in the EXAMPLE.

In some embodiments, the method of the present invention comprises the step of comparing the determined level of phenylalanine with a predetermined reference value. Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. The full name of ROC curve is receiver operator characteristic curve, which is also known as receiver operation characteristic curve. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis is. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, when the determined level of phenylalanine is higher than the predetermined reference value it is concluded that the subject has or is at risk of having a cardiac dysfunction.

The method of the present invention is particularly suitable for the early diagnosis of cardiac dysfunction. As used herein the term “early diagnosis” refers to an early phase of establishing the existence or degree of cardiac dysfunction in the subject, before a symptom or a group of symptoms appears.

Thus the method of the present invention is particularly suitable for prescribing a therapy suitable for preventing the development of cardiac dysfunction.

In some embodiment, the cardiac dysfunction is cardiac senescence.

In some embodiment, the cardiac senescence is premature cardiac senescence

Thus, the invention also refers to a method for early diagnosis of cardiac senescence comprising determining the level of phenylalanine in sample obtained from the subject wherein said level indicates whether the subject has cardiac senescence.

A further object of the present invention relates to use of phenylalanine as a biomarker of cardiac dysfunction.

A further object of the present invention relates to use of phenylalanine as a biomarker of cardiac senescence.

In some embodiment, the cardiac senescence is premature cardiac senescence.

A further object of the present invention relates to a method of determining whether a patient achieves a response with a drug that is used for the treatment of cardiac dysfunction in a patient comprising determining the level of phenylalanine in a sample obtained from the patient during the course of the treatment wherein an increase in said level indicates that the patient does not achieve a response or wherein a stable level or a decreased level indicates that the patient achieves a response.

In some embodiment, the cardiac dysfunction is cardiac senescence.

In some embodiment, the cardiac senescence is premature cardiac senescence.

The method is thus particularly suitable for discriminating responder from non-responder. As used herein the term “responder” in the context of the present disclosure refers to a patient that will achieve a response, i.e. a patient where cardiac dysfunction is reduced or improved. According to the invention, the responders have an objective response and therefore the term does not encompass patients having a stabilized cardiac dysfunction such that the disease is not progressing after the therapy. A non-responder or refractory patient includes patients for whom cardiac dysfunction does not show reduction or improvement after the therapy. According to the invention the term “non-responder” also includes patients having a stabilized cardiac dysfunction. Typically, the characterization of the patient as a responder or non-responder can be performed by reference to a standard or a training set. The standard may be the profile of a patient who is known to be a responder or non-responder or alternatively may be a numerical value. Such predetermined standards may be provided in any suitable form, such as a printed list or diagram, computer software program, or other media. When it is concluded that the patient is a non-responder, the physician could take the decision to stop the therapy to avoid any further adverse sides effects or to adjust the dose for improving the response.

A further object of the present invention relates to a method of treating cardiac dysfunction in a patient in need thereof comprising administering to the patient a therapeutically effective agent that is capable of increasing the catabolism of phenylalanine (i.e. promoting phenylalanine degradation), whereby lowering phenylalanine levels.

In some embodiments, the subject is considered as having or is at risk of having cardiac dysfunction as determined by the diagnostic method as above described.

In some embodiment, the cardiac dysfunction is cardiac senescence.

Thus, the present invention relates to a method of treating cardiac senescence in a patient in need thereof comprising administering to the patient a therapeutically effective agent that is capable of increasing the catabolism of phenylalanine (i.e. promoting phenylalanine degradation), whereby lowering phenylalanine levels.

In some embodiment, the cardiac senescence is premature cardiac senescence.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the therapeutic method of the present invention is particularly suitable for the prophylactic treatment of cardiac dysfunction in elderly patients, and/or obese patients and/or patients who exhibit one or more risk factors for cardiac dysfunction (e.g. age, alcohol consumption, cigarette smoking, metabolic syndrome, obesity, diabetes/insulin resistance, hypertension, dyslipidaemia, live disease or chronic kidney disease) and/or patients suffering from any form of acquired and hereditary hyperphenylalaninemia, including phenylketonuria (PKU).

In some embodiment, the cardiac dysfunction is cardiac senescence.

In some embodiment, the cardiac senescence is premature cardiac senescence.

The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

In some embodiments, the agent is capable of reactivating phenylalanine hydroxylase (PAH).

In some embodiments, the agent is BH4. As used herein, the term “BH4” has its general meaning in the art and refers to tetrahydrobiopterin (THB) also known as sapropterin. BH4 is a cofactor of PAH. The IUAPC name is 2-amino-6-(1,2-dihydroxypropyl)-5,6,7,8-tetrahydro-3H-pteridin-4-one. BH4 in the form of a dihydrochloride salt is commercially available under the trade name Kuvan®.

By a “therapeutically effective amount” of the agent as above described is meant a sufficient amount to provide a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the agent is administered to the subject in the form of a pharmaceutical composition. Typically, the agent may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Accordingly, pH may be adjusted to a value where the agent delivered is stable or suitable adjuvants, such as antioxidants or other stabilisers, are added to prevent premature demise of said agent. Solutions comprising compounds of the invention as free base or pharmacologically acceptable salts can be prepared in water suitably mixed with a surfactant, such as hydroxypropylcellulose or cyclodextrins. Dispersions can also be prepared in glycerol, liquid polyethylene glycols, and mixtures thereof and in oils. Under ordinary conditions of storage and use, these preparations contain a preservative to prevent the growth of microorganisms. The agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Phenylalanine (PA) induces senescence. a, Immunoblot of indicated markers of senescence in C2C12 cells treated with PA (5 mM) or vehicle with quantification (n=6/group). b, Immunoblot of p21 as a function of PA concentration with quantification (n=4/concentration). c, p21 protein levels after treatment with PA (5 mM), BH4 (10 μM) and 4-CPA (1.5 mM) with quantification as indicated (n=4/condition). d, Quantification of EdU incorporation in C2C12 cells after treatment with PA, BH4 and 4-CPA as indicated (n=4/condition). e, Cytosolic superoxide levels (with CellROX probe) in C2C12 cells as a function of PA concentration (n=4/concentration). f, Cytosolic superoxide levels as treated with PA, BH4 and 4-CPA as indicated (n=4/condition). g, Immunoblot of p21 in primary adult rat cardiomyocytes treated with PA (5 mM) and/or BH4 (10 μM) vs. vehicle. Data are presented as original immunoblot images (a-c) and mean±SEM analysed with ANOVA with Bonferroni post-hoc test (a-f); ns: non-significant, *p<0.05, **p<0.01, ***p<0.001 as indicated.

FIG. 2. p21 deficiency protects against senile molecular, structural and functional changes in the myocardium. a, Plasma PA levels in 2 and 15 month-old WT and p21-/- mice (n=9-10/group). b, Immunoblot analysis of hearts of 2- and 15-month-old WT mice (n=3/age). c, p21 colocalisation with cardiomyocyte marker troponin I and fibroblast marker vimentin, but not CD31 in aged WT hearts. d, Percentage of 4-HNE-positive area in hearts of WT and p21-/- mice at indicated ages (n=4/condition). e, Representative immunofluorescent images of Pah and 2-SC (and merge) in hearts of WT and p21-/- mice at indicated ages (n=3/condition). f, Representative immunofluorescent images of Pah and 2-SC (and merge) in human hearts of indicated ages. g, Heart weight to tibia length (HW/TL) of WT and p21-/- mice at indicated ages (n=8-11/condition). h, Quantification of myocardial interstitial fibrosis (Sirius red) of WT and p21-/- mice at indicated ages (n=4/condition). i-k, Systolic strain rate (i), dP/dtₘₐₓ (j) & dP/dtₘᵢₙ (k) of WT and p21-/- mice at indicated ages (n=8-11/condition). For microscopic images in panel c, magnification: 400×, scale-bar: 50 μm, in panels e-f: 200× scale-bar: 100 μm. Data are presented as original images (b-c, e-f) or mean±SEM analysed with ANOVA with Bonferroni post-hoc test (a, d, g-k); ns: non-significant, *p<0.05, **p<0.01, ***p<0.001 as indicated.

FIG. 3. Phenylalanine (PA) is a driver of cardiac ageing. a, Schematic of treatment and evaluation protocol. b, Plasma PA levels in vehicle- or PA-treated WT mice of 6 and 12 months of age (n=8-11/condition). c, Heart weight to tibia length (HW/TL) for the same groups (n=8-11/condition). d, Representative images of hearts stained with wheat-germ agglutinin (WGA) Sirius red, vimentin for the same groups (n=4/condition). e-g, Quantification of cardiomyocyte cross-sectional area (CSA; e; n=4/group), interstitial fibrosis (Sirius red, f; n=4/group) and vimentin-positive areas (g; n=4/group) in hearts of the same animals as above. h-k, LV ejection fraction (h), systolic strain rate (i), dP/dtₘₐₓ (j) & dP/dtₘᵢₙ (k; n=8-11/condition) in the same animals as above. l, Representative microscopic images of myocardial p21, Pah, Gch1, 2-SC & 4-HNE in the same animals as above (n=3-4/condition). For all microscopic images, magnification: 200×, scale-bar: 50 μm. Data are presented either as original images (d & l) or as mean±SEM analysed with ANOVA with Bonferroni post-hoc test (b-c, e-k); ns: non-significant, *p<0.05, **p<0.01, ***p<0.001 as indicated.

FIG. 4. BH4 rescues plasma PA levels and senile cardiac deterioration by enhancing hepatic PA catabolism. a, Plasma PA levels in 12.5 month-old WT mice treated with intraperitoneal BH4 or vehicle (n=8-10/group). b-f, Heart weight to tibia length (b; HW/TL), systolic strain rate (c), dP/dtₘₐₓ (d) & dP/dtₘᵢₙ (e) and arterial pressures (f; n=9-10/group) in mice treated as above. g-h, Quantification of myocardial Sirius red (g) and vimentin staining (h) (n=4/group). i, Myocardial nitric oxide synthase activity in 12.5 month-old WT mice treated with intraperitoneal BH4 or vehicle (NOS; n=9-10/group). j, Immunofluorescence of p21, Pah, Gch1 and 2-SC as above (n=4/group). k, Representative Pah and Gch1 immunofluorescent images of livers in 2 and 15 month-old WT and p21-/- mice (n=3/group). l, Immunoblots of senescence markers 2- and 15-month-old WT as indicated (n=³/ₐge). m, Hepatic phenylalanine content after subcutaneous PA administration or with increased age (n=9-10/group). n, Immunoblot of p21 from AML-12 hepatocytes as a function of PA (0-10 mM) added. o, Immunoblots of vehicle (VEH)- and Nutlin3a (NU)-treated AML-12 hepatocytes with p21 or scrambled siRNA (scr; n=3/condition). p-q, Tyrosine levels in media of AML-12 cells treated with vehicle or Nutlin3a and p21 or scrambled siRNA (p) and treatment with Nutlin3a without or with BH4 (q; n=5-6/group). r, Pah immunoblot in AML-12 cells treated with Nutlin3a+/−BH4 (n=3/group). s, Representative immunofluorescent images of liver Pah in vehicle- and BH4-treated 12.5-month-old WT mice (n=3/group). t, Hepatic PA content in 12.5-month-old WT mice treated with BH4 or vehicle (n=9-10/group). u, Standardized expression of PAH against age in biopsies from liver donors (n=33). For microscopic images, magnification: 200×, scale-bar: 50 μm. Data are presented as original images (j-l, n-o, r-s) or mean±SEM and analysed with two-tailed unpaired t-test (a-i, q, t), ANOVA with Bonferroni post-hoc test (m and p) or linear regression analysis (u); ns: non-significant, *p<0.05, **p<0.01, ***p<0.001.

### EXAMPLE

### Methods

### Animal Husbandry

Procedures involving animals were approved by the Institutional Animal Care and Use Committee of the French National Institute of Health and Medical Research (INSERM)-Unit 955, Créteil, France (ComEth 15-001). Global p21-/- mice backcrossed to C57BL6 background for at least 10 generations (Jackson) as well as wild-type (WT) littermates were kept in individually ventilated cages in a high-health facility with 12-hour light-dark cycle, controlled temperature (20-22° C.) and humidity. Water and chow were provided ad libitum.

### Cardiac Phenotyping

Male WT and p21-/- mice were followed from the age of 2 to 15 months of age. These mice were sequentially evaluated for myocardial structure and function. Animals were euthanized and tissues harvested for histology and molecular biology (at ages 2, 6, 10 and 15 months). A separate group of p21-/- mice (n=17) were allowed to age further. These mice displayed an inconspicuous phenotype with no mortality until at least 24 months (not shown).

### In Vivo Drug Treatment

PA in a dose of 200 mg/kg twice a day or vehicle (1×PBS) was subcutaneously administered to 11 month-old WT mice (Janvier Labs, France) in vivo for a month. BH4 in a dose of 10 mg/kg/die 2×/die or vehicle (1×PBS with 10 mM sodium ascorbate and citric acid to pH 4.5) was intraperitoneally administered to 11 month-old WT mice in vivo over six weeks. General state of the mice (body weight & wellbeing) was closely monitored. In both cases drug treatment was completed as scheduled without incidents.

### 2D Transthoracic Echocardiography in Conscious Mice

Mice were trained to be grasped for transthoracic echocardiography (TTE) that was performed in non-sedated mice to avoid the cardiac depressor effect of anesthetic agents, as previously reported.¹ Heart rates at recordings were typically above 600 beats per minute (bpm).

Data acquisition for a single cohort was performed by a single operator (JT or ER). Images were acquired from a parasternal position at the level of the papillary muscles using a 13-MHz linear-array transducer with a digital ultrasound system (Vivid 7, GE Medical System, Horton, Norway). Left ventricular dimensions and ejection fraction, anterior and posterior wall thicknesses were serially obtained from M-mode acquisition. Relative LV wall thickness (RWT) was defined as the sum of septal and posterior wall thickness over LV end-diastolic diameter, and LV mass was determined using the uncorrected cube assumption formula (LV mass=(AWTd+LVEDD+PWTd)³−(LVEDD)³). Peak systolic values of radial strain rate of the anterior and posterior wall were obtained using Tissue Doppler Imaging (TDI) as previously described.² TDI loops were acquired from parasternal view with a careful alignment with the radial component of the deformation' at a mean frame rate of 514 fps and a depth of 1 cm. The Nyquist velocity limit was set at 12 cm/s. Radial strain rate analysis was performed offline using the EchoPac Software (GE Medical Systems) blindly by a single operator (GD). Peak systolic of radial strain rate was computed from a region of interest positioned in the mid anterior wall and was measured over an axial distance of 0.6 mm. The temporal smoothing filters were turned off for all measurements. Because of the inevitable respiratory variability, we averaged peak systolic of radial strain rate on 8 consecutive cardiac cycles.

### Invasive In Vivo Haemodynamic Assessment of Left Ventricular Function

In vivo haemodynamic measurements were performed just before mice of indicated ages were sacrificed.' Haemodynamic evaluation was performed in mice placed on a homeothermic operating table in supine position under 1.5% isoflurane anesthesia with spontaneous breathing. A 1.4-Fr microcatheter (Millar Instruments, Houston, USA) was calibrated manually before each experiment, inserted via the right carotid artery into the aorta and subsequently advanced to the left ventricle. Data were collected after at least 10 min of baseline, using the lowest isoflurane concentration tolerated to ensure minimal cardiodepression during measurement of peak rates of isovolumetric pressure development (dP/dtₘₐₓ) and pressure decay (dP/dtₘᵢₙ). The microcatheter was then withdrawn to the aorta for measurement of systolic and diastolic pressure. Data were analyzed using the IOX software (EMKA, France).

### Tissue Harvesting and Processing

All mice were weighed, euthanized by cervical dislocation, followed by rapid excision of the organs of interest. The heart was cannulated through the aorta for perfusion with ice-cold 1×PBS, then blotted and weighed. The heart was cut in half perpendicular to its axis: the apical two-third was snap-frozen in liquid nitrogen, whilst the basic one-third was recannulated through the aorta, perfused with 10% formalin for histology. Hearts were kept in formalin at 4° C. for at least 24-48 hours before embedding. Snap-frozen tissues were kept at −80° C., until they were powdered using a mortar and pestle cooled with liquid nitrogen and collected as aliquots. Livers and kidneys were processed in a similar manner.

### Human Heart Biopsies

Human heart biopsies (right atrial appendage) were obtained from patients undergoing elective coronary artery bypass grafting. Biopsies were obtained after approval of the ethical committee (Comité de Protection des Personnes Ile-de France VI) of Pitié-Salpêtrière Hospital, Paris and informed consent was acquired from each patient prior to the procedure.⁴

### Human Liver Data

BioMart (https://www.ncbi.nlm.nih.gov/pubmed/14707178) was used to map microarray probesets to the human assembly in Ensembl release 96. The available human liver transcriptomic data was generated in 33 non-diseased, beating heart liver donors with Affymetrix GeneChip Human Genome U133 Plus 2.0 Arrays (https://www.ncbi.nlm.nih.gov/pubmed/29554203), which had 3 probesets that mapped to PAH.⁵ The annotations and normalized data were downloaded from NCBI Gene Expression Omnibus (accession number GSE107039). Principal components analysis in IBM SPSS Statistics v25 was used to reduce the data to a standardized expression of PAH, which was regressed as a dependent variable against donor age in GraphPad Prism v8.

### Expression Profiling Across Human Tissues

The expression data from the Genotype-Tissue Expression Project (http://gtexportal.org/, GTEx) (https://www.ncbi.nlm.nih.gov/pubmed/23715323) reflects 16,000 samples taken from multiple tissues across 752 donors (65% male, age 20-79) within 24 hours of death and quantified by RNAseq. The median TPM by tissue analysis V7 was downloaded, underwent log 10 transformation, and visualized with Java TreeView (https://www.ncbi.nlm.nih.gov/pubmed/15180930).^{6,7}

### Senescence-Associated β-galactosidase Staining

Senescence-associated β-galactosidase activity was used to estimate global cardiac senescence. Briefly, a section of freshly harvested hearts was incubated for 1 hour at 37° C. in β-galactosidase staining solution containing 1 mg/ml X-Gal (Sigma), 40 mM citric acid, 150 mM NaCl, 2 mM MgCl₂, 5 mM potassium ferrocyanide and 5 mM potassium ferricyanide with the pH adjusted to 6.0. Stained sections were then scanned.

### Isolation and Culture of Ventricular Primary Adult Rat Cardiomyocytes

Male Spargue Dawley rats (9 weeks, 300-350 g) were anesthetised with ketamine and xylazine (100 and 10 mg/kg, respectively) with heparin added (100 UI/kg). Hearts were excised and retrogradely perfused with an oxygenated (95% CO₂, 5% O₂) perfusion buffer consisting of NaCl 113 mM, KCl 4.7 mM, KH₂PO₄ 0.6 mM, Na₂HPO₄ 0.6 mM, MgSO₄-7H₂O 1.2 mM, NaHCO₃ 12 mM, KHCO₃ 10 mM, HEPES 10 mM, Taurine 30 mM, phenol red 0.032 mM, D-glucose 5.5 mM, 2,3-butanedionemonoxime 10 mM, pH 7.4) for 2 minutes to wash out the blood from the coronary arteries. Then hearts were perfused with a digestion buffer (perfusion buffer supplemented with 0.1 mg/mL liberase, 0.14 mg/mL trypsine-EDTA and 12.5 μM Ca²⁺) for 10-12 minutes. Hearts were then placed in a stopping buffer (perfusion buffer supplemented with 10% NBCS and 12.5 μM Ca²⁺). Atria and right ventricles were removed. Left ventricles were dissected into small fragments, then subjected to successive aspirations-reflux. Digested ventricles were filtered through a 250 μm cell strainer. After 10 minutes of incubation at 37° C., supernatants were discarded and cells were resuspended with a calcium buffer (perfusion buffer supplemented with 5% NBCS, 12.5 μM Ca²⁺). Extracellular calcium was added incrementally up to 1 mM. Finally, cells were suspended in culture medium M199 (supplemented with 1% ITS), seeded on wells pre-coated with 10 μg/mL laminin and allowed to attach for 2 h before starting treatments. Cardiomyocytes were subsequently treated with 5 mM PA with or without BH4 against vehicle. After a 4-hour incubation cardiomyocytes were snap-frozen and transferred to −80° C. for protein work conducted a few days later. At the time of harvesting rod shape, the presence of cross-striations and the absence of vesicles on their surface were visually confirmed to insure viable status of cardiomyocytes.

### RNA Work and Quantitative RT-PCR

Total RNA from cells or powdered tissue was extracted using the RNeasy mini kit or RNeasy mini fibrous tissue kit (QIAGEN), respectively, as previously described.⁴ RNA yield was measured with Nanodrop ND-1000, a >1.9 260/280 ratio was accepted. For reverse transcription the High Capacity cDNA kit was used according to the manufacturer's instructions (Applied Biosystems). Quantitative reverse transcriptase-polymerase chain reaction (qRT-PCR) was used to quantify relative levels of genes of interest. Briefly, amplification reactions were carried out using Fast Universal Master Mix on a StepOnePlus system (Applied Biosystems) in a multiplexed design (FAM: gene of interest; VIC: β-actin as endogenous control) in a reaction volume of 10 μl. All Taqman oligos used were inventoried Taqman-MGB oligos from Applied Biosystems. Relative expression was quantified using the ΔC_{T} method with the formula RQ=2^{-ΔΔCT} (User Bulletin #2; Applied Biosystems).⁴

### Protein Extraction and Western Blotting

Cells were washed briefly with ice-cold 1×PBS, scraped in 400 μL T-PER lysis buffer (ThermoFisher) supplemented with 0.1 mM phenylmethylsulphonyl fluoride (PMSF) and protease/phosphatase inhibitor cocktail tablet (ThermoFisher). Pulverised tissue samples (20-30 mg) were lysed in the same buffer accelerated by sonication and passing through a 21 G needle. To pellet any debris, lysates were centrifuged at 10,000 g for 10 minutes at 4° C., with the supernatant transferred to fresh tubes. Protein concentrations were determined with Bradford assay (Biorad) and lysates were diluted to equal concentration with lysis buffer. Protein lysates were then mixed with Laemmli buffer, vortexed and heated to 95° C. for 5 min. Equal amounts of protein lysates in parallel with protein molecular weight marker (ThermoFisher) were loaded onto pre-cast polyacrylamide gels (Nupage 10 or 12% Bis Tris gel, Novex, Invitrogen) and separated by electrophoresis at 200V for 1 hour. Proteins were transferred onto polyvinylidene difluoride (PVDF) membrane (Invitrogen) using an electrophoretic transfer cell (Mini Trans-Blot, Bio-Rad) in ice-cold transfer buffer (48 mM Tris base, 390 mM glycine, 0.1% SDS, 20% methanol (v/v)) at 300 mA for 2 hours. Membranes were blocked in 1× skimmed milk (ThermoFisher) for 1 hour at room temperature, followed by overnight incubation with primary antibody diluted in blocking buffer at 4° C. The antibodies and concentrations used are shown in Table 2. Next day membranes were washed, followed by 60 minutes incubation in the corresponding horseradish peroxidase (HRP)-conjugated secondary antibody diluted 1:2000-10000 in blocking buffer (all Abcam). Blots were developed using enhanced chemiluminescence (ECL) reagents (normal, Prime & Select) and digital images were acquired using an imaging system (Azure Biosystems). Membranes were usually stripped of antibodies and reprobed with an antibody targeting a protein serving as loading control (α-actinin for hearts & β-actin for all others). For this procedure, membranes were placed in Guanidine-HCl-based stripping solution (6M Guanidine-HCl, 0.2% Nonidet P-40, 0.1M β-mercaptoethanol, 20 mM Tris-HCl, pH 7.5 and 100 mM 2-mercaptoethanol) and incubated for 10 minutes at room temperature with gentle agitation, followed by extensive washing and re-blocking.⁵

TABLE 1
Primary antibodies used in the study.

| Target | | | Application/ |
|---|---|---|---|
| protein | Producer/Cat# | Species | concentration |
| p21 | Abcam: ab188224 | rabbit | WB: 1:1000 |
| p21 | Abcam: ab80633 | mouse | IF: 1:200 |
| p21 | Santa Cruz: sc-397 | rabbit or | IF: 1:50 |
| | | goat | |
| p-p53 | Cell Signaling: 9284S | rabbit | WB: 1:1000 |
| p53 | Cell Signaling: 2524S | mouse | IF: 1:200 |
| p16 | Abbiotec: 250804 | rabbit | IF: 1:100, WB: 1:100 |
| β-actin | Abcam: ab49900 | mouse- | WB: 1:10000 |
| | | HRP | |
| Pah | Sigma: SAB250434 | goat | IF: 100, WB: 1:200 |
| Pah | Abcam: ab148430 | rabbit | IHC: 1:200, IF: 1:100, |
| | | | WB: 1:200-1000 |
| Gch1 | Bioss antibodies: | rabbit | IF: 100, WB: 1:200- |
| | bs-0136R | | 1000 |
| 2-SC | Cambridge BioSciences: | rabbit | IHC/IF: 1:100, WB: |
| | crb2005017e | | 1:200 |
| Nrf2 | Genetex: GTX103322 | rabbit | WB: 1:500 |
| 4-HNE | Millipore: AB5605 | goat | IHC: 1:200 |
| vimentin | Abcam: ab92547 | rabbit | IF: 1:200 |
| α-actinin | Abcam: ab68167 | rabbit | WB: 1:5000 |
| CD31 | Santa Cruz: sc-18916 | rat | IF: 1:200 |
| cardiac | Abcam: ab47003 | rabbit | IF: 1:200 |
| troponin I | | | |

IHC: immunohistochemistry, IF: immunofluorescence, WB: Western blot.

### Histology

Formalin-fixed organs were embedded in paraffin. Cross-sections were cut into 7 μm thickness using a rotary microtome (Leica). To assess cardiac fibrosis, Sirius red staining was performed, followed by dehydration and mounting with Eukitt quick-hardening mounting medium (Sigma). To visualise cardiomyocyte hypertrophy, sections were incubated in 2 μg/mL Texas red-conjugated wheat germ agglutinin (WGA; Invitrogen) in 1×PBS at room temperature for 45 minutes. Slides were then mounted with fluorescent mounting media (Abcam). Images were acquired using a Zeiss fluorescent microscope. Whole-mount preparations were made of hearts and livers of 2- and 15-month-old p21-mCherry reporter mice. Briefly, organs were quickly removed and 1-mm-thick sections were cut with a custom-made chamber equipped with razor blades. Subsequently, sections were laid out on a slide, soaked in 1×PBS and mounted with a cover slip for fluorescent microscopic evaluation.

### Immunohistochemistry/Immunofluorescence

Whenever a dispersed pattern was expected, immunohistochemistry was performed on paraffin-embedded sections. Briefly, after rehydration, citrate buffer-assisted, heat-mediated antigen retrieval and blocking, sections were incubated overnight at +4° C. with primary antibodies raised against the following targets: 4-HNE (Millipore; goat, 1:200), Pah (Abcam; rabbit, 1:200) or 2-SC (Cambridge Biosciences; rabbit, 1:100; also see Table 1). Next day sections were washed, then incubated with the corresponding HRP-conjugated secondary antibodies (Abcam; 1:200) for 30 minutes at room temperature, washed again, followed by incubation with 3,3′-diaminobenzidine (DAB; Sigma) under visual observation until the signal appeared. Then slides were either counterstained with haematoxylin or not, dehydrated and mounted.

Immunofluorescence was used for higher sensitivity and co-localisation of proteins of interest. Briefly, paraffin-embedded sections were rehydrated, followed by antigen retrieval with citrate-assisted heat. Sections were then blocked with 30% goat serum (using antibody dilution solution with background reducing components; Dako) or Bloxall™ artificial blocking agent (Dako) and incubated overnight at +4° C. with primary antibodies raised against target proteins (see Table 1 for details). The following day sections were washed in 1×TBST, incubated with a mixture of the corresponding Alexa Fluor 555-labeled secondary antibodies (Invitrogen) and Alexa-488-conjugated Phalloidin (to counterstain cardiomyocytes) for 30 minutes at room temperature, and washed again. In case of co-labelling Alexa Fluor 555-labelled and Alexa Fluor 647-labeled secondary antibodies were used (Invitrogen), occasionally in combination with the avidin/biotin enhancement system (Dako). Specific protocols are available upon request. Slides were mounted with fluorescent mounting media with DAPI (4′,6-diamidino-2-phenylindole; Abcam). Images were obtained with a Zeiss confocal microscope.

### Cell Culture

C2C12 (ATCC® CRL-1772™) myoblasts maintained in DMEM supplemented with L-glutamine (2 mM), penicillin/streptomycin (1%) and 5% fetal bovine serum (FBS) were seeded either at a density of 5×10⁴ cells (<50%) per well in 24-well plates or 10⁴ cells in 4-well chamber slides. Cells were with L-phenylalanine (1-10 mM)+BH4 (10 μM) and incubation with increasing concentrations of N-acetylcysteine (0.5, 2.5, 5 mM) vs. vehicle overnight as indicated. Cells were analysed for oxidative stress with MitoSOX™ (mitochondrial superoxide; 5 μM, ThermoFisher Scientific), CellROX™ (cytosolic superoxide; 5 μM, ThermoFisher Scientific), gene or protein expression, or various metabolites (see below).

AML12 cells (ATCC® CRL-2254™) were maintained in DMEM/F-12 (1:1) culture media supplemented with 10% FBS, 10 μg/ml insulin, 5.5 μg/ml transferrin, 5 ng/ml selenium, 40 ng/ml dexamethasone and 1% penicillin/streptomycin. Cells were sub-cultivated from subconfluent flasks with a ratio from 1:4 to 1:8. For WB analysis, cells were plated on 6-well plates and harvested at 80-90% of confluence. AML12 cells were transfected with indicated siRNA (Table 2) at 80% confluence using Lipofectamine RNAiMAX Transfection Reagent (ThermoFischer Scientific 13778030) following the manufacturer's instructions. Transfection efficiency was constantly checked using BLOCK-IT Alexa Fluor Red Fluorescent Control (ThermoFischer Scientific 14750100).

All cell culture experiments were performed in biological triplicates at the least.

TABLE 2
siRNA used for transfection experiments.

| Target mRNA | Producer/Cat# |
|---|---|
| Cdkn1a (p21) | ThermoFischer Scientific: 60538 |
| Scrambled | ThermoFischer Scientific: 12935112 |

### Determination of Myocardial Nitric Oxide Synthase Activity

Myocardial nitric oxide synthase (NOS) activity was determined in hearts of 12.5-month-old WT mice treated with BH4 or vehicle using a commercial kit (Biovision). Briefly, pulverized heart aliquots were processed according to the manufacturer's instructions and fluorometric readouts (excitation: 360, emission: 450) were normalised to protein concentration determined by Bradford assay. Activity of recombinant NOS protein served as positive control.

### Determination of Metabolite Levels

Levels of reduced glutathione (GSH; ThermoFisher), phenylalanine (BioVision) and tyrosine (BioVision) were determined from cells or plasma, with respective agents/kits following the manufacturer's recommendations. Specifically, GSH levels were estimated with the aid of ThiolTracker™ Violet fluorescent probe (ThermoFisher). Plasma and liver phenylalanine levels were determined using an enzyme-coupled, fluorometric method (BioVision). Briefly, samples were deproteinized via trichloroacetate precipitation, neutralised, tyrosinase-treated (to remove tyrosine that may interfere) and diluted to fit in the standard curve. Fluorescence reading took place at 587 nm after excitation at 535 nm. Levels of tyrosine in cells or conditioned media were determined after deproteinization using 10 kDa cut-off columns with an enzyme-coupled, colorimetric method with reading at 491 nm (BioVision).

### Data Analysis and Statistics

Mice were randomly assigned to experimental groups and data were acquired and analysed blind to genotype, age or treatment. Statistical analyses were performed using GraphPad Prism Software (version 6). In all cases n numbers were raised to obtain Gaussian distribution and parametric tests were used. Accordingly, Student's t-test was used to compare two groups, whilst more than two groups were compared using one-way analysis of variance (ANOVA), with Bonferroni post-hoc test for multiple comparisons. Two-way ANOVA was used to compare groups with time-dependent evolution of readouts, with Bonferroni post-hoc test for more than two groups. Data are presented as mean±standard error of the mean. Annotations used: *p<0.05, **p<0.01, ***p<0.001 compared to groups indicated. A p value of <0.05 was considered significant.

### REFERENCES

1. Ternacle J, Wan F, Sawaki D, Surenaud M, Pini M, Mercedes R, Ernande L, Audureau E, Dubois-Rande J L, Adnot S, Hue S, Czibik G, Derumeaux G. Short-term high-fat diet compromises myocardial function: a radial strain rate imaging study. Eur Heart J Cardiovasc Imaging. 2017.

2. Derumeaux G, Ichinose F, Raher M J, Morgan J G, Coman T, Lee C, Cuesta J M, Thibault H, Bloch K D, Picard M H, Scherrer-Crosbie M. Myocardial alterations in senescent mice and effect of exercise training: a strain rate imaging study. Circulation. Cardiovascular imaging. 2008; 1(3):227-234.

3. Ferferieva V, Van den Bergh A, Claus P, Jasaityte R, La Gerche A, Rademakers F, Herijgers P, D'Hooge J. Assessment of strain and strain rate by two-dimensional speckle tracking in mice: comparison with tissue Doppler echocardiography and conductance catheter measurements. European heart journal cardiovascular Imaging. 2013; 14(8): 765-773.

4. Sawaki D, Czibik G, Pini M, Ternacle J, Suffee N, Mercedes R, Marcelin G, Surenaud M, Marcos E, Gual P, Clement K, Hue S, Adnot S, Hatem S N, Tsuchimochi I, Yoshimitsu T, Henegar C, Derumeaux G. Visceral Adipose Tissue Drives Cardiac Aging Through Modulation of Fibroblast Senescence by Osteopontin Production. Circulation. 2018.

5. Bacalini M G, Franceschi C, Gentilini D, Ravaioli F, Zhou X, Remondini D, Pirazzini C, Giuliani C, Marasco E, Gensous N, Di Blasio A M, Ellis E, Gramignoli R, Castellani G, Capri M, Strom S, Nardini C, Cescon M, Grazi G L, Garagnani P. Molecular Aging of Human Liver: An Epigenetic/Transcriptomic Signature. J Gerontol A Biol Sci Med Sci. 74(1):1-8.

6. Moore HM. Acquisition of normal tissues for the GTEx program. Biopreserv Biobank. 11(2):75-76.

7. The Genotype-Tissue Expression (GTEx) project. Nat Genet. 45(6):580-585.

8. Ashrafian H, Czibik G, Bellahcene M, Aksentijevic D, Smith A C, Mitchell S J, Dodd M S, Kirwan J, Byrne J J, Ludwig C, Isackson H, Yavari A, Stottrup N B, Contractor H, Cahill T J, Sahgal N, Ball D R, Birkler R I, Hargreaves I, Tennant D A, Land J, Lygate C A, Johannsen M, Kharbanda R K, Neubauer S, Redwood C, de Cabo R, Ahmet I, Talan M, Gunther U L, Robinson A J, Viant M R, Pollard P J, Tyler D J, Watkins H. Fumarate is cardioprotective via activation of the Nrf2 antioxidant pathway. Cell Metab. 2012; 15(3):361-371.

9. Yeung Y G, Stanley E R. A solution for stripping antibodies from polyvinylidene fluoride immunoblots for multiple reprobing. Anal Biochem. 2009; 389(1):89-91.

### Results and Discussion

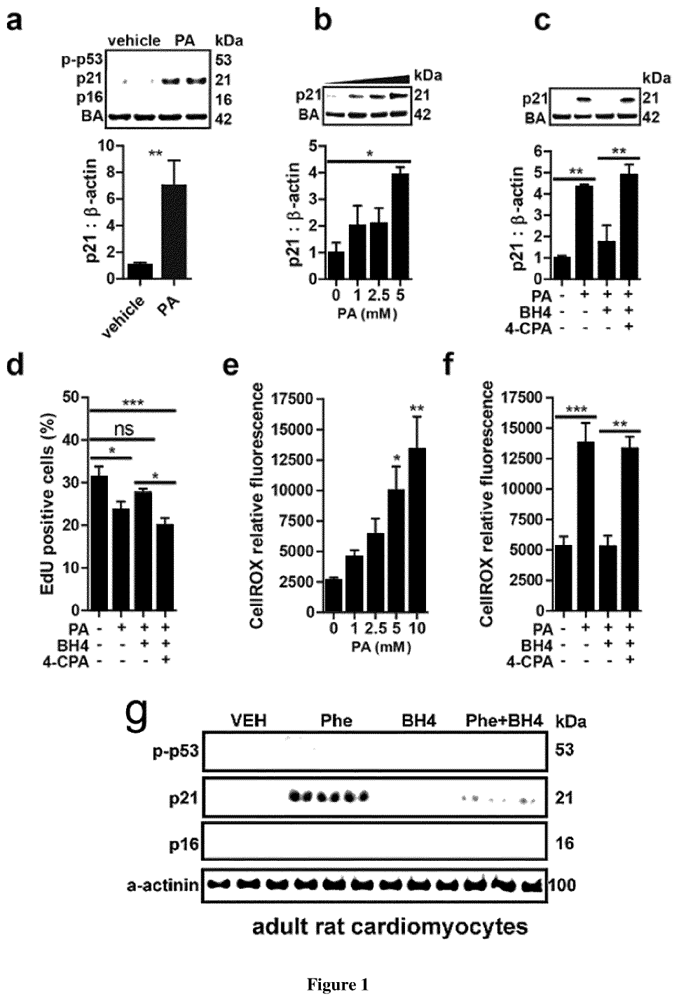

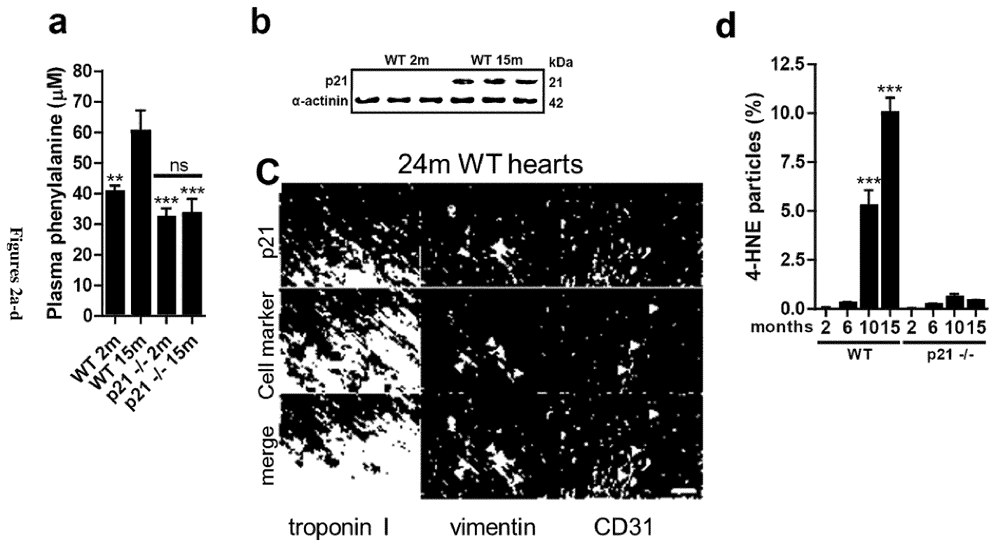

To assess the role of PA in cellular senescence in vitro, we treated C2C12 myoblasts with PA⁹. PA selectively induced cyclin-dependent kinase 1a (Cdkn1a=p21) and suppressed EdU incorporation, whilst other markers of senescence (Cdkn2a=p16 and phospho-p53=p-p53) remained unaltered (FIG. 1a-d). BH4 antagonised PA-dependent senescence, which was reversed by PA analogue 4-chlorophenylalanine (4-CPA; FIG. 1c-d). BH4 stimulated PA catabolism, as evidenced by increased cellular levels of Pah, tyrosine and 2-succinylcysteine (2-SC; data not shown). BH4-dependent succination, a spontaneous posttranslational modification between PA catabolite fumarate and cysteine, came along with activation of the succination-sensitive Nrf2-antioxidant pathway (data not shown).¹⁰ Interestingly, PA incrementally increased cytosolic, but not mitochondrial superoxide levels (FIG. 1e & data not shown). BH4 normalized superoxide levels, a rescue prevented by 4-CPA (FIG. 1f). This raised the question whether PA induces senescence through oxidative stress.¹¹ To resolve this matter, we used the antioxidant N-acetylcysteine (NAC), which like BH4, restored normal cytosolic superoxide and reduced glutathione (GSH; data not shown) levels, yet failed to prevent p21 induction (data not shown), suggesting an oxidative stress-independent role for PA in triggering cellular senescence. Finally, we confirmed the selective induction of p21 protein (but not that of p53 and p16) and its rescue by BH4 in primary adult rat cardiomyocytes (FIG. 1g). Collectively, our experiments establish the link between PA and cellular senescence by p21 induction, motivating in vivo studies.

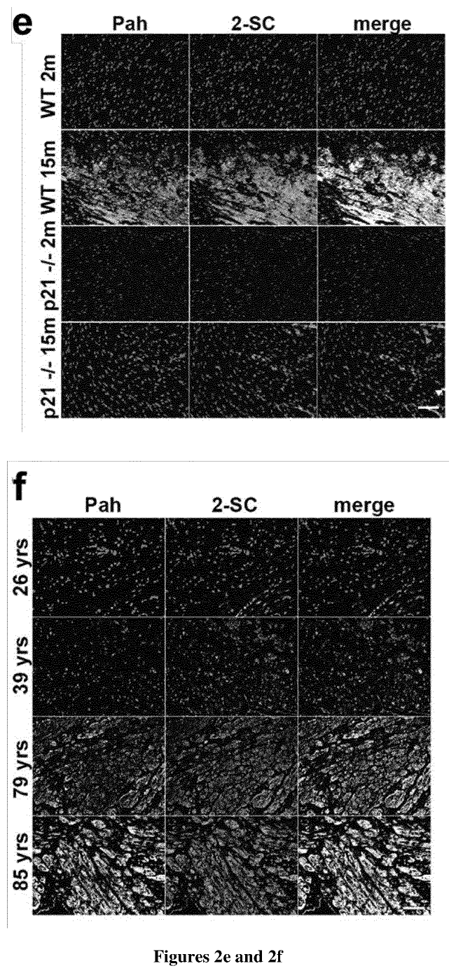

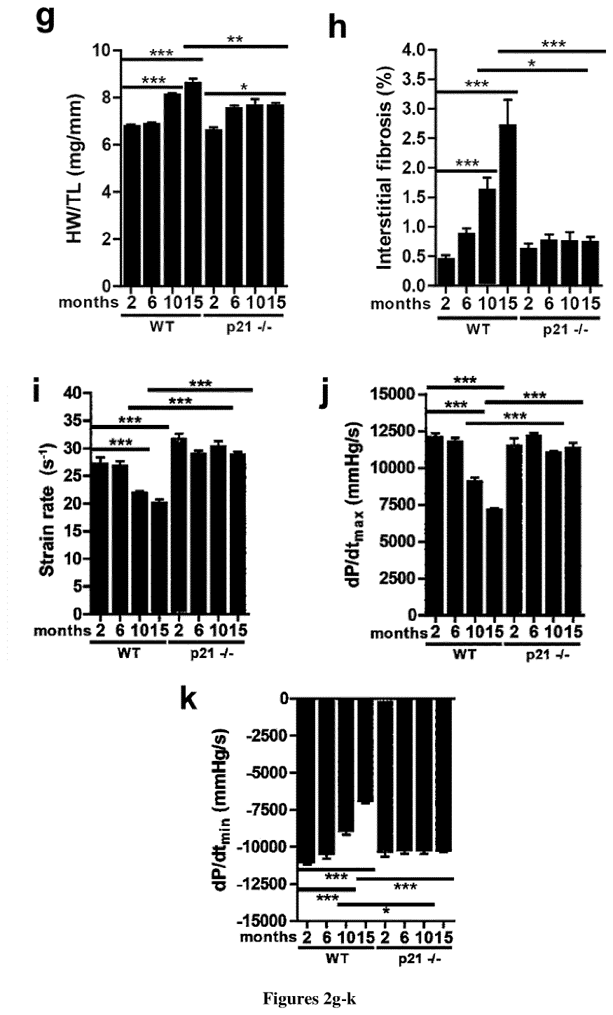

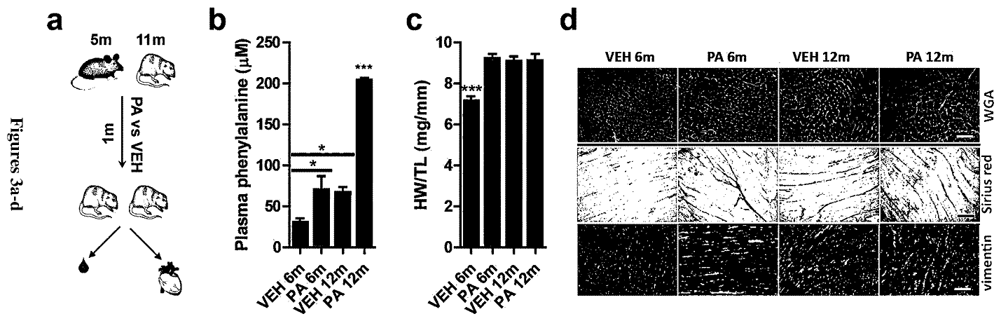

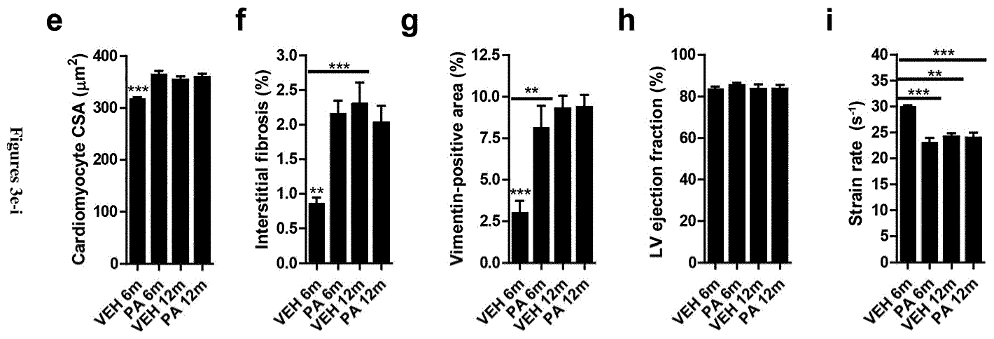

Based on our in vitro findings showing PA activating p21 together with reports indicating that p21 deficiency improves lifespan¹², we focused our efforts on p21-/- mice. As reported in humans,^{13, 14} plasma PA levels increased in wild-type mice (WT) with age (2 vs. 15 months, an age with senile cardiac alterations),^{2, 3} whilst p21-/- mice were protected from senile rise in plasma PA levels (FIG. 2a).

With chronological ageing, p21 and senescence-associated β-galactosidase, but not p53 and p16 expression increased in WT myocardium (FIG. 2b & data not shown). Induction of p21 protein colocalised with cardiomyocyte marker troponin I and fibroblast marker vimentin, but not endothelial cell marker CD31 in aged WT hearts (FIG. 2c). In addition, 4-hydroxynonenal (4-HNE), a marker of lipid peroxidation with cytosolic presence'^{s} also increased with age in hearts of WT but not p21-/- mice (FIG. 2d & data not shown). Ageing progressively upregulated and co-localized Pah and 2SC in WT hearts, but p21 deficiency prevented this (FIG. 2e & data not shown). Notably, human hearts presented with a comparable induction and colocalization of Pah and 2SC with age (FIG. 2f). Similarly, the rate-limiting enzyme of de novo BH4 biosynthesis GTP cyclohydrolase 1 (Geh1) was upregulated in old WT but not in p21-/- hearts, as were transcriptional targets of Nrf2, a sensitive responder to succination¹⁰ (data not shown).

The observed increase in plasma PA and cardiac Pah levels were associated with cardiac hypertrophy and myocardial interstitial fibrosis in aged WT mice (FIG. 2g-h & data not shown). By contrast, aged p21-/- mice did not show any cardiac remodeling in line with normal plasma PA levels. Furthermore, whilst left ventricular ejection fraction remained normal (data not shown), diastolic (dP/dtₘᵢₙ) and systolic dysfunction (dP/dtₘₐₓ, systolic strain rate) were unmasked by in vivo haemodynamics and advanced echocardiographic deformation imaging in aged WT but not p21-/- mice (FIG. 2i-k). Collectively, p21 deficiency protected against molecular, structural and functional changes of chronologically ageing myocardium while preventing increase in plasma PA levels.

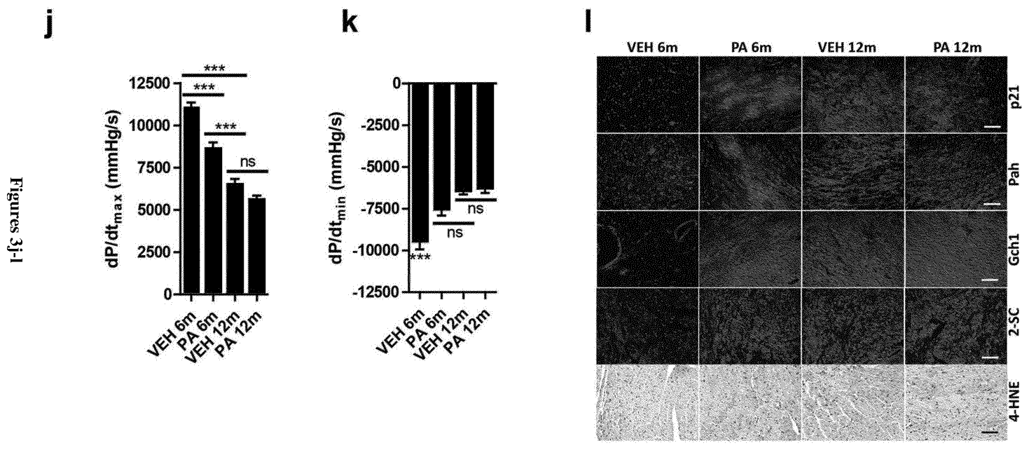

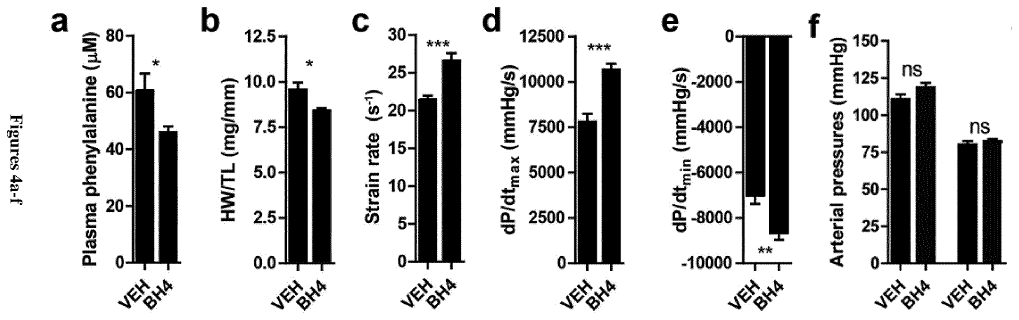

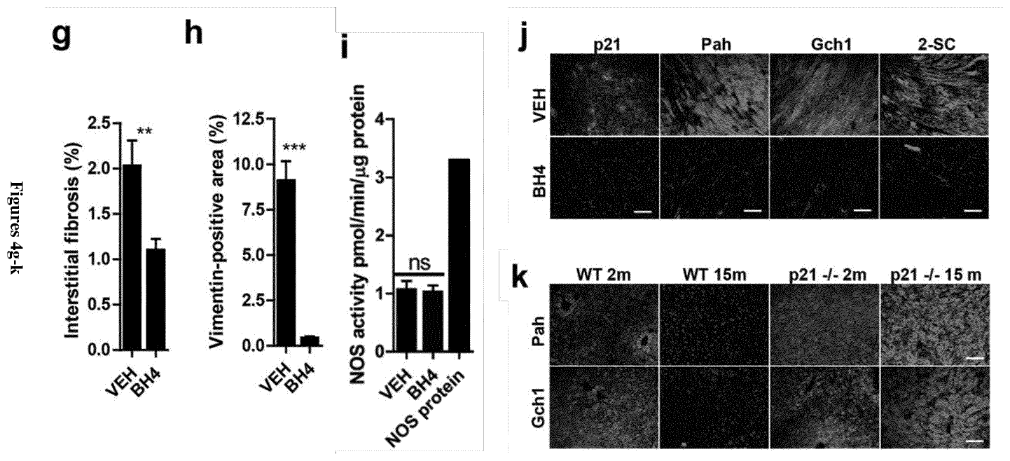

To establish a direct role for PA in driving cardiac ageing, we decided to treat WT mice without and with senile myocardium (see FIG. 2b-k; i.e. 5 vs. 11 month-old) at a dose of PA 200 mg/kg twice a day over one month with the intent to increase plasma PA levels (FIG. 3a). Mice tolerated PA treatment well without significant weight loss (data not shown). Plasma PA levels increased in both age-groups, but were considerably higher in older animals (FIG. 3a). Young animals with PA treatment exhibited a senile-like phenotype with an increase in cardiac hypertrophy, cardiomyocyte size and myocardial interstitial fibrosis, whilst no change occurred in the older group (FIG. 3c-g). At unaltered ejection fraction, PA treatment compromised cardiac function in the young group with reduced strain rate, dP/dtₘₐₓ and dP/dtₘᵢₙ (FIG. 3h-k) reaching the range of vehicle-treated old mice. PA triggered myocardial senescence with upregulation of p21, Pah, Gch1, 2-SC and 4-HNE in young hearts indistinguishable from old ones (FIG. 3l & data not shown). These findings clearly demonstrate that excess PA induces cardiac senescence in young mice, closely mimicking senile cardiac remodeling and dysfunction.³

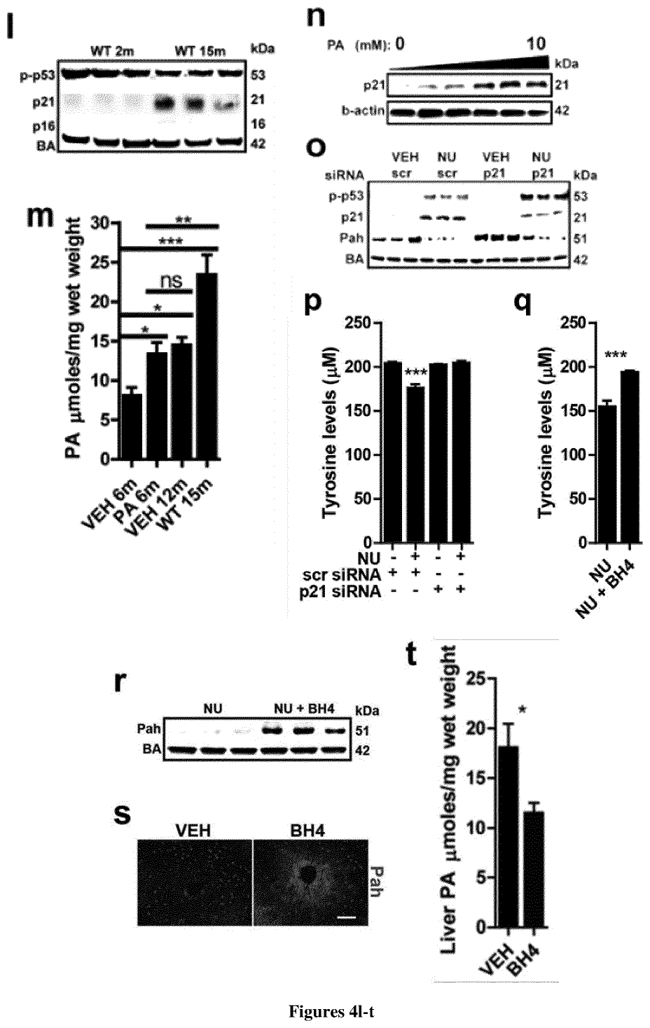

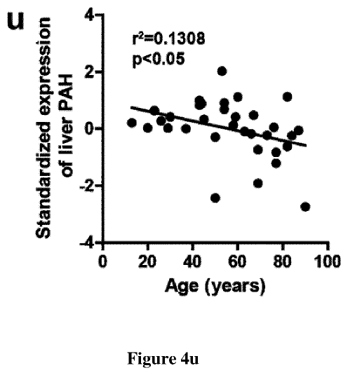

Since PA induces cardiac ageing, we speculated that PA catabolism may restore cardiac function in older mice. Accordingly, 11-month-old WT mice were intraperitoneally injected with 10 mg/kg BH4 twice a day over 6 weeks to enhance Pah activity and reduce plasma PA to young levels (FIG. 4a). BH4 ameliorated HW/TL, contractility and relaxation to young values, without lowering systemic arterial pressures (FIG. 4b-f).^{16, 17} Moreover, BH4 normalised interstitial fibrosis whilst suppressing senescent myocardial expression of p21, Pah, Gch1 & 2-SC without altering myocardial nitric oxide synthase activity (FIG. 4g-i & data not shown).

Restored plasma PA levels along with repressed myocardial Pah, Gch1 and 2-SC by BH4 turned our attention to natural Pah expressors, kidney and liver. Whilst no age-related downregulation of Pah and Gch1 occurred in kidney, we uncovered depressed protein levels of Pah and Gch1 in 15-month-old WT livers, which were prevented by p21 deficiency (FIG. 4k & data not shown). Since ageing induced liver senescence (by p21 expression; FIG. 4l & data not shown), we hypothesized that senescence undermines hepatic PA catabolism. To this end first we explored hepatic PA levels, finding an age-dependent increase in WT livers (FIG. 4m). Interestingly, livers of PA-treated 6-month-old WT mice had comparable hepatic PA levels to those of 12-month-old WT mice (FIG. 4m). From the findings of age-dependent increase in hepatic PA content and p21 induction we hypothesized that excess PA induces p21. To address this possibility we treated AML-12 hepatocytes with increasing concentrations of PA and found a dose-dependent induction of p21 protein (FIG. 4n). Next, we treated AML-12 hepatocytes with p53 activator Nutlin3a in order to indirectly induce p21 activity. Nutlin3a induced hepatocyte senescence (i.e. p-p53 and p21), downregulated Pah and depressed tyrosine production (FIG. 4o-p & data not shown). Knockdown with p21 siRNA in hepatocytes restored Pah levels as well as tyrosine production (FIG. 4o-p). Importantly from a translational point of view, BH4 derepressed tyrosine and Pah levels from Nutlin3a-induced senescence (FIG. 4q-r) in vitro and in vivo re-expressed Pah in liver (FIG. 4s), improved hepatic Pah activity (data not shown) and reduced hepatic PA content (FIG. 4t).^{18, 19} Moreover, human relevance of an age-dependent decline in hepatic Pah activity is demonstrated by an age-dependent reduction in hepatic Pah transcript levels based on reanalysis of RNAseq data of a cohort of non-diseased liver biopsies (FIG. 4u).²⁰ In summary, our findings highlight PA toxicity in age-related cardiac decline and opens new avenues to rejuvenate aged hearts by pharmacologically restoring hepatic Pah activity.

Taken together, elevated PA levels have a previously overlooked impact in cardiac ageing. Our results suggest that myocardium takes its share in maintaining homeostasis by catabolising excess PA. Whether evolving cardiac PA catabolism along with signaling consequences,²¹ accumulating toxic metabolites,^{22, 23} or PA-fueled catecholamine biosynthesis²⁴ accounts more for cardiac ageing needs further exploring. Our findings pointed to failing hepatic PA catabolism behind rising PA levels. Pah has by far the highest hepatic expression of the six BH4-dependent enzymes (aromatic amino acid hydroxylases & nitric oxide synthases; data not shown). Its vital dependence on BH4 is illustrated by the observation that hyperphenylalaninemia is a prominent feature in enzymatic deficiencies in de novo BH4 biosynthesis or recycling pathway BH4²⁵. Accordingly, in naturally aged mice plasma PA levels were restored by portal BH4, consistent with revived hepatic Pah activity.^{18, 19}

Therapeutic exploitation of amino acid metabolism has been demonstrated via forced histidine catabolism to sensitise cancers to methotrexate.²⁶ According to the findings presented here, pharmacologically restored PA catabolism by BH4 or alternative means²⁷ makes reversal of cardiac ageing a realistic aim. Further age-related states may also benefit from Pah reactivation, such as dementia²⁸ and susceptibility to cancer.^{29, 30} Finally, PKU patients, especially those abandoning PKU diet later in life, may be at higher cardiovascular risk.³¹

### REFERENCES

Throughout this application, various references describe the state of the art to which this invention pertains. The disclosures of these references are hereby incorporated by reference into the present disclosure.

1. Lakatta E G, Levy D. Arterial and cardiac aging: major shareholders in cardiovascular disease enterprises: Part II: the aging heart in health: links to heart disease. Circulation. 2003; 107(2):346-354.

2. Derumeaux G, Ichinose F, Raher M J, Morgan J G, Coman T, Lee C, Cuesta J M, Thibault H, Bloch K D, Picard M H, Scherrer-Crosbie M. Myocardial alterations in senescent mice and effect of exercise training: a strain rate imaging study. Circ Cardiovasc Imaging. 2008; 1(3):227-234.

3. Sawaki D, Czibik G, Pini M, Ternacle J, Suffee N, Mercedes R, Marcelin G, Surenaud M, Marcos E, Gual P, Clement K, Hue S, Adnot S, Hatem S N, Tsuchimochi I, Yoshimitsu T, Henegar C, Derumeaux G. Visceral Adipose Tissue Drives Cardiac Aging Through Modulation of Fibroblast Senescence by Osteopontin Production. Circulation. 2018.

4. Ternacle J, Wan F, Sawaki D, Surenaud M, Pini M, Mercedes R, Ernande L, Audureau E, Dubois-Rande J L, Adnot S, Hue S, Czibik G, Derumeaux G. Short-term high-fat diet compromises myocardial function: a radial strain rate imaging study. Eur Heart J Cardiovasc Imaging. 2017.

5. Eriksson J G, Guzzardi M A, Iozzo P, Kajantie E, Kautiainen H, Salonen M K. Higher serum phenylalanine concentration is associated with more rapid telomere shortening in men. Am J Clin Nutr. 105(1):144-150.

6. Delles C, Rankin N J, Boachie C, McConnachie A, Ford I, Kangas A, Soininen P, Trompet S, Mooijaart S P, Jukema J W, Zannad F, Ala-Korpela M, Salomaa V, Havulinna A S, Welsh P, Wurtz P, Sattar N. Nuclear magnetic resonance-based metabolomics identifies phenylalanine as a novel predictor of incident heart failure hospitalisation: results from PROSPER and FINRISK 1997. Eur J Heart Fail. 2018; 20(4):663-673.

7. Schuck P F, Malgarin F, Cararo JH, Cardoso F, Streck E L, Ferreira G C. Phenylketonuria Pathophysiology: on the Role of Metabolic Alterations. Aging Dis. 2015; 6(5):390-399.

8. Park S K, Kim K, Page G P, Allison D B, Weindruch R, Prolla T A. Gene expression profiling of aging in multiple mouse strains: identification of aging biomarkers and impact of dietary antioxidants. Aging Cell. 2009; 8(4):484-495.

9. Sanayama Y, Matsumoto A, Shimojo N, Kohno Y, Nakaya H. Phenylalanine sensitive K562-D cells for the analysis of the biochemical impact of excess amino acid. Sci Rep. 4:6941.

10. Ashrafian H, Czibik G, Bellahcene M, Aksentijevic D, Smith A C, Mitchell S J, Dodd M S, Kirwan J, Byrne J J, Ludwig C, Isackson H, Yavari A, Stottrup N B, Contractor H, Cahill T J, Sahgal N, Ball D R, Birkler R I, Hargreaves I, Tennant D A, Land J, Lygate C A, Johannsen M, Kharbanda R K, Neubauer S, Redwood C, de Cabo R, Ahmet I, Talan M, Gunther U L, Robinson A J, Viant M R, Pollard P J, Tyler D J, Watkins H. Fumarate is cardioprotective via activation of the Nrf2 antioxidant pathway. Cell Metab. 2012; 15(3):361-371.

11. Chen W, Sun Z, Wang X J, Jiang T, Huang Z, Fang D, Zhang D D. Direct interaction between Nrf2 and p21(Cip1/WAF1) upregulates the Nrf2-mediated antioxidant response. Mol Cell. 2009; 34(6):663-673.

12. Choudhury A R, Ju Z, Djojosubroto M W, Schienke A, Lechel A, Schaetzlein S, Jiang H, Stepczynska A, Wang C, Buer J, Lee H W, von Zglinicki T, Ganser A, Schirmacher P, Nakauchi H, Rudolph K L. Cdkn1a deletion improves stem cell function and lifespan of mice with dysfunctional telomeres without accelerating cancer formation. Nat Genet. 2007; 39(1):99-105.

13. Cynober L, Blonde F, Nguyen Dinh F, Gerbet D, Giboudeau J. [Measurement of plasma and urinary amino acids with gas chromatography in healthy subjects. Variations as a function of age and sex]. Ann Biol Clin (Paris). 1983; 41(1):33-38.

14. Bancel E, Strubel D, Bellet H, Polge A, Peray P, Magnan de Bornier B. [Effect of the age and the sex on plasma concentration of amino acids]. Ann Biol Clin (Paris). 1994; 52(9): 667-670.

15. Al Nimer F, Strom M, Lindblom R, Aeinehband S, Bellander B M, Nyengaard JR , Lidman O, Piehl F. Naturally occurring variation in the Glutathione-S-Transferase 4 gene determines neurodegeneration after traumatic brain injury. Antioxid Redox Signal. 18(7):784-794.

16. Cunnington C, Van Assche T, Shirodaria C, Kylintireas I, Lindsay A C, Lee J M, Antoniades C, Margaritis M, Lee R, Cerrato R, Crabtree M J, Francis J M, Sayeed R, Ratnatunga C, Pillai R, Choudhury R P, Neubauer S, Channon K M. Systemic and vascular oxidation limits the efficacy of oral tetrahydrobiopterin treatment in patients with coronary artery disease. Circulation. 2012; 125(11):1356-1366.

17. Hashimoto T, Sivakumaran V, Carnicer R, Zhu G, Hahn V S, Bedja D, Recalde A, Duglan D, Channon K M, Casadei B, Kass D A. Tetrahydrobiopterin Protects Against Hypertrophic Heart Disease Independent of Myocardial Nitric Oxide Synthase Coupling. J Am Heart Assoc. 2016; 5(3):e003208.

18. Thony B, Ding Z, Martinez A. Tetrahydrobiopterin protects phenylalanine hydroxylase activity in vivo: implications for tetrahydrobiopterin-responsive hyperphenylalaninemia. FEB S Lett. 2004; 577(3):507-511.

19. Kure S, Sato K, Fujii K, Aoki Y, Suzuki Y, Kato S, Matsubara Y. Wild-type phenylalanine hydroxylase activity is enhanced by tetrahydrobiopterin supplementation in vivo: an implication for therapeutic basis of tetrahydrobiopterin-responsive phenylalanine hydroxylase deficiency. Mol Genet Metab. 2004; 83(1-2):150-156.

20. Bacalini M G, Franceschi C, Gentilini D, Ravaioli F, Zhou X, Remondini D, Pirazzini C, Giuliani C, Marasco E, Gensous N, Di Blasio A M, Ellis E, Gramignoli R, Castellani G, Capri M, Strom S, Nardini C, Cescon M, Grazi G L, Garagnani P. Molecular Aging of Human Liver: An Epigenetic/Transcriptomic Signature. J Gerontol A Biol Sci Med Sci. 74(1):1-8

21. Ternette N, Yang M, Laroyia M, Kitagawa M, O'Flaherty L, Wolhulter K, Igarashi K, Saito K, Kato K, Fischer R, Berquand A, Kessler B M, Lappin T, Frizzell N, Soga T, Adam J, Pollard P J. Inhibition of mitochondrial aconitase by succination in fumarate hydratase deficiency. Cell Rep. 3(3):689-700.

22. Fernandes C G, Leipnitz G, Seminotti B, Amaral A U, Zanatta A, Vargas C R, Dutra Filho C S, Wajner M. Experimental evidence that phenylalanine provokes oxidative stress in hippocampus and cerebral cortex of developing rats. Cell Mol Neurobiol. 2010; 30(2):317-326.

23. Rosa A P, Jacques C E, Moraes T B, Wannmacher C M, Dutra Ade M, Dutra-Filho C S. Phenylpyruvic acid decreases glucose-6-phosphate dehydrogenase activity in rat brain. Cell Mol Neurobiol. 2012; 32(7):1113-1118.

24. Ziegler MG , Lake C R, Kopin I J. Plasma noradrenaline increases with age. Nature. 1976; 261(5558):333-335.

25. Werner E R, Blau N, Thony B. Tetrahydrobiopterin: biochemistry and pathophysiology. Biochem J. 2011; 438(3):397-414.

26. Kanarek N, Keys H R, Cantor J R, Lewis C A, Chan S H, Kunchok T, Abu-Remaileh M, Freinkman E, Schweitzer L D, Sabatini D M. Histidine catabolism is a major determinant of methotrexate sensitivity. Nature. 2018.

27. Isabella V M, Ha B N, Castillo M J, Lubkowicz D J, Rowe S E, Millet Y A, Anderson C L, Li N, Fisher A B, West K A, Reeder P J, Momin M M, Bergeron C G, Guilmain S E, Miller P F, Kurtz C B, Falb D. Development of a synthetic live bacterial therapeutic for the human metabolic disease phenylketonuria. Nat Biotechnol 36(9):857-864.

28. Ravaglia G, Forti P, Maioli F, Bianchi G, Martelli M, Talerico T, Servadei L, Zoli M, Mariani E. Plasma amino acid concentrations in patients with amnestic mild cognitive impairment or Alzheimer disease. Am J Clin Nutr. 2004; 80(2):483-488.

29. DeNicola G M, Karreth F A, Humpton T J, Gopinathan A, Wei C, Frese K, Mangal D, Yu K H, Yeo C J, Calhoun E S, Scrimieri F, Winter J M, Hruban R H, Iacobuzio-Donahue C, Kern S E, Blair I A, Tuveson D A. Oncogene-induced Nrf2 transcription promotes ROS detoxification and tumorigenesis. Nature. 2011; 475(7354):106-109.

30. Wu L E, Gomes A P, Sinclair D A. Geroncogenesis: metabolic changes during aging as a driver of tumorigenesis. Cancer Cell 25(1):12-19.

31. Hermida-Ameijeiras A, Crujeiras V, Roca I, Calvo C, Leis R, Couce M L. Arterial stiffness assessment in patients with phenylketonuria. Medicine (Baltimore). 2017; 96(51):e9322.

32. Shinmura Ken. Cardiac Senescence, Heart Failure, and Frailty : A triangle in elderly people. J Med. 2016 Jun. 25; 65(2):25-32.

1. A method of predicting whether a subject has or is at risk of having a cardiac dysfunction comprising determining the level of phenylalanine in a sample obtained from the subject wherein said level indicates whether the subject has or is at risk of having a cardiac dysfunction.

2. The method of claim 1 wherein the subject exhibits one or more risk factors for cardiac dysfunction or is a subject who does not exhibit risk factors, or is a subject who is asymptomatic for cardiac dysfunction.

3. The method of claim 1 wherein the subject is an elderly subject.

4. The method of claim 1 wherein the subject is obese.

5. The method of claim 1 wherein the subject suffers from a form of acquired and hereditary hyperphenylalaninemia.

6. The method of claim 1 which comprises a step of comparing the determined level of phenylalanine with a predetermined reference value.

7. The method of claim 6 wherein when the determined level of phenylalanine is higher than the predetermined reference value it is concluded that the subject has or is at risk of having a cardiac dysfunction.

8. The method of claim 1, wherein the cardiac dysfunction is cardiac senescence.

9. The method of claim 8, wherein the cardiac senescence is premature cardiac senescence.

10. (canceled)

11. (canceled)

12. A method of determining whether a patient achieves a response with a drug that is used for the treatment of cardiac dysfunction in a patient, comprising determining the level of phenylalanine in a sample obtained from the patient during the course of the treatment wherein an increase in said level indicates that the patient does not achieve a response or wherein a stable level or a decreased level indicates that the patient achieves a response.

13. A method of preventing or treating cardiac dysfunction in a patient in need thereof comprising administering to the patient a therapeutically effective agent that is capable of increasing the catabolism of phenylalanine, thereby lowering phenylalanine levels.

14. The method of claim 13 wherein the method is performed prophylactically to prevent cardiac dysfunction in elderly patients, and/or obese patients and/or patients who exhibit one or more risk factors for cardiac dysfunction and/or patients suffering from acquired and hereditary hyperphenylalaninemia.

15. The method of claim 11 wherein the agent is BH4.

16. A method of preventing or treating cardiac dysfunction in a subject in need thereof, comprising
determining the level of phenylalanine in a sample obtained from the subject, and, when the level is higher than a previously determined reference value,
administering to the subject a therapeutically effective amount of an agent that increases the catabolism of phenylalanine.

17. The method of claim 16, wherein the agent is BH4.

18. The method of claim 2 wherein the one or more risk factors include age, alcohol consumption, cigarette smoking, metabolic syndrome, obesity, diabetes/insulin resistance, hypertension, dyslipidaemia, liver disease and chronic kidney disease.

19. The method of claim 5 wherein the subject suffers from phenylketonuria (PKU).

20. The method of claim 14 wherein the one or more risk factors include age, alcohol consumption, cigarette smoking, metabolic syndrome, obesity, diabetes/insulin resistance, hypertension, dyslipidaemia, liver disease or chronic kidney disease.

21. The method of claim 14 wherein the subject suffers from phenylketonuria (PKU).
